(19) United States
(12) Patent Application Publication
(10) Pub. No.: US 2025/0228666 A1
(43) Pub. Date: Jul. 17, 2025

(54) CONICALLY EXPANDABLE MITRAL VALVE REPAIRMENT DEVICE

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); NIRAVKUMAR MAHESHBAI RANA, Valsad (IN)

(73) Assignee: MERIL HEALTHCARE PVT. LTD, Vapi (IN)

(21) Appl. No.: 18/697,650

(22) PCT Filed: Aug. 5, 2022
(86) PCT No.: PCT/IN2022/050706

§ 371 Date: Apr. 1, 2024

(30) Foreign Application Priority Data
Dec. 17, 2021 (IN) 202121059026

(51) Int. Cl. A61F 2/24

(52) U.S. Cl. CPC A61F 2/2466; A61F 2/2454

(57) ABSTRACT

An implant (100) for treatment of regurgitation in a mitral valve (100) is disclosed. The implant (100) comprising at least one fastener (50) including a top portion (52) and a shank (54). The top portion (52) including a ring (51a) and a plurality of crowns (60). The crowns (60) include a primary arm (61) and a secondary arm (63), the primary arm (61) being longer than the secondary arm (63). The ring (51a) is attached at a proximal end (51) of the shank (54) and a nut (53a) is attached at a distal end (53) of the shank (54). The primary arm (61) is attached to the ring (51a) and the secondary arm (63) is attached to the nut (53a). The top portion (52) is rotated in a predefined direction leading to upward movement of the nut (53a) in order to actuate the implant (100).

### FIELD OF INVENTION

The present invention relates to a medical device. More specifically, the present invention relates to a medical device for repairing a mitral valve.

### BACKGROUND

Human heart has four valves namely, a tricuspid valve, a bicuspid valve (mitral valve), an aortic valve and a pulmonary valve. A bicuspid valve is also known as the mitral valve. The mitral valve is positioned between the left atrium and the left ventricle and has two flaps. Mitral regurgitation is the most critical condition in which the leaflets of the mitral valve do not close enough resulting in abnormal backward blood flow from the left ventricle to the left atrium. In mitral regurgitation, the left ventricle is unable to consume blood as per the volume of the ventricle while excessive blood flows back into the left atrium.

There are various causes leading to mitral valve regurgitation such as valve prolapse, damaged tissue cord, rheumatic fever, endocarditis, abnormality of heart muscle, heart failure, etc. Treatment of mitral valve regurgitation depends on severity of the condition. Generally, treatment of mild leakage is performed by drugs dedicated to improve functioning of the heart. However, in case of acute leakage, a surgery may be performed.

Conventionally, for cardiac operations, open heart surgeries are performed. Such operations typically involve cutting and opening the chest of a patient (e.g., via a median sternotomy or a thoracotomy approach). These procedures can be painful and very invasive, and often lead to medical complications and also have a slow recovery time. In addition, patients who are in poor medical condition may not be eligible to receive open heart surgery due to the risks associated with such operations, thereby preventing the much-needed surgical treatment of the heart disease.

A transcatheter based approach is used for the surgery which may reduce the operation time.

In such approaches, regurgitation in mitral valve can be treated by repairing or reshaping the valve leaflets of the native mitral valve. The mitral valve can be repaired by replacing supportive cords and/or removing excessive valve tissue, so that the leaflets can close tightly thereby, reducing valve regurgitation. The reshaping of the mitral valve (annular reduction) can be performed by attaching an artificial annuloplasty ring with the annulus. Alternatively, a clip may be used to close the leaflets. Such devices may involve critical procedure which may take longer time duration.

Or, a resizable device may also be used to resize and/or reshape the mitral valve. One such implant is disclosed in an application U.S. Pat. No. 9,180,005B1. However, the implant disclosed in the said application may present several limitations, for example, more time consumption for actuation of the implant at the implantation site which may lead to increase in risks and complications associated with the surgery.

Therefore, there exists a need for an improved implant which can overcome limitations of the conventional ones.

### SUMMARY

The present invention relates to an implant for treatment of regurgitation in a mitral valve is disclosed. The implant comprising at least one fastener including a top portion and a shank. The top portion including a ring and a plurality of crowns. The crowns include a primary arm and a secondary arm, the primary arm being longer than the secondary arm. The ring is attached at a proximal end of the shank and a nut is attached at a distal end of the shank. The primary arm is attached to the ring and the secondary arm is attached to the nut. The top portion is rotated in a predefined direction leading to upward movement of the nut in order to actuate the implant.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an implant 100 in accordance with an embodiment of the present invention.

FIG. 1a depicts a fastener 50 of the implant 100 in accordance with an embodiment of the present invention.

FIG. 1b depicts a crown 60 of the implant 100 in accordance with an embodiment of the present invention.

FIG. 2 depicts a flow chart of a process involved in manufacturing of the implant 100 in accordance with an embodiment of the present invention.

FIG. 3 depicts a delivery device 300 in accordance with an embodiment of the present invention.

### DETAILED DESCRIPTION OF DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a medical implant (implant) is disclosed. The implant is used to treat mitral valve regurgitation in a patient. However, it should be noted that the application of the implant of the present invention may also be extended to treat the tricuspid valve regurgitation in a patient as well.

The implant may include at least one fastener, a slotted nut, multiple crowns and anchors coupled together in a predefined manner enabling conical expansion of the implant. The crowns may be arranged in an L shaped structure. The L shaped structure of the crowns facilitates equal stress distribution on both the arms while actuation thereby preventing damage to the tissue wall.

The implant may be a conically expandable implant. The implant may be used to treat annular regurgitation of the mitral valve in case the size of the native annulus is between 28 mm to 40 mm.

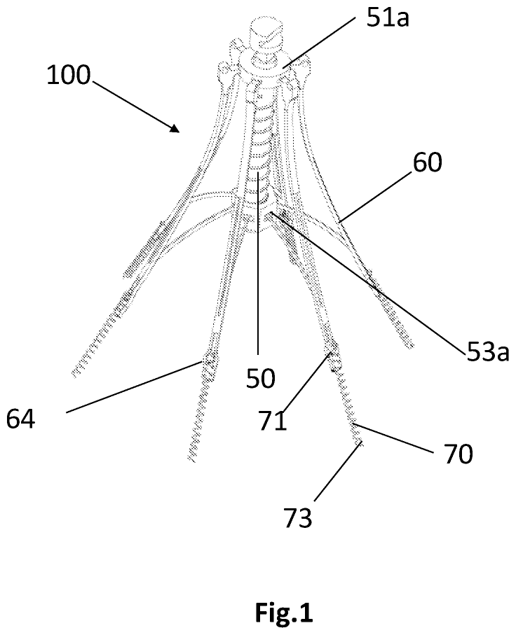

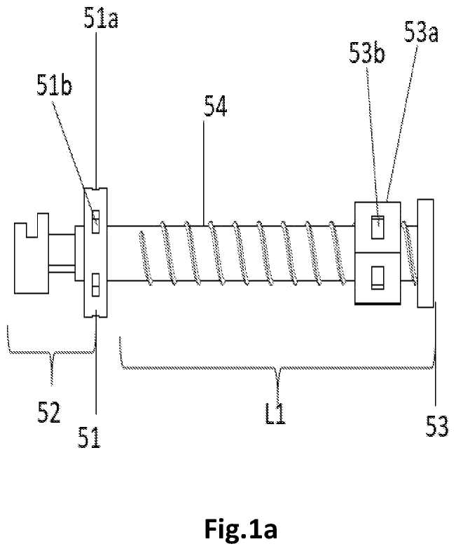

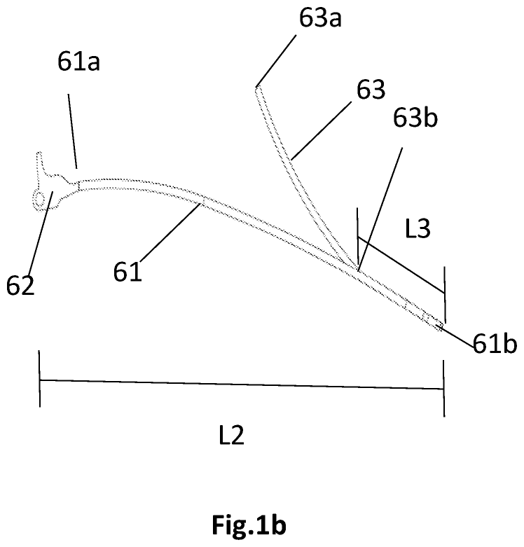

Now moving specifically to drawings, FIG. 1 depicts an implant 100. The implant 100 may be deployed at an annulus of a mitral valve in order to reshape and/or reconnect leaflets of the mitral valve, thereby reducing annular diameter and regurgitation of the mitral valve.

The implant 100 is illustrated in the conical expandable configuration. The implant 100 may include one or more components coupled with each other in a predefined manner. The implant 100 may be used in the mitral valve where the diameter of the native annulus is more than 25 mm.

The implant 100 may include but not limited to at least one fastener 50, a plurality of crowns 60, and a plurality of anchors 70. These components of the implant 100 are designed to work together as a functional unit, to repair a stenosed mitral valve in a patient.

The fastener 50 may be made of but not limited to 316L SS, 316LVM SS, 304 SS, Nitinol. In an embodiment, the fastener 50 is made of 316L SS.

The fastener 50 (as illustrated in FIG. 1a) may include a top portion 52 and a shank 54 including a proximal end 51, a distal end 53 and a length L1 extending between the proximal end 51 and the distal end 53. The length L1 maybe in a range of 10 mm to 30 mm. In an embodiment, the length L1 is 20 mm. The top portion 52 of the fastener 50 includes a ring 51a attached at the shank 54 as explained in paragraphs below.

The shank 54 may include a predefined outer diameter ranging from 1 mm to 5 mm. In an embodiment, the outer diameter is 3 mm. The shank 54 may be threaded. The shank 54 may include a predefined pitch ranging from 0.3 mm to 0.8 mm. In an embodiment, the pitch of the threads of the shank 54 is 0.5 mm.

The fastener 50 may include at least one slot (not shown) disposed at each of the proximal end 51 and the distal end 53. The slot present at the proximal end 51 of the shank 54 may be used to attach the ring 51a of the top portion 52 and the slot present at the distal end 53 of the shank 54 may be used to attach a nut 53a as depicted in FIG. 1. The top portion 52 of the implant 100 may be rotated in a predefined direction in order to actuate the implant 100 at the implantation site. In an embodiment, the top portion 52 is rotated in the clockwise direction. The rotation of the top portion 52 may trigger upward movement of the nut 53a resulting in radial contraction of the crowns 60 leading to actuation of the implant 100.

The ring 51a may be fixedly attached on the shank 54 by means of welding. The nut 53a may be removably screwed to the shank 54. The ring 51a and the nut 53a may include a plurality of first grooves 51b and a plurality of second grooves 53b respectively. The plurality of grooves 51b and 53b may be used to attach the crown 60 as explained below. The plurality of grooves 51b and 53b may be in a range of 5 to 10. In an embodiment, the ring 51a and the nut 53a include six groves 51b and 53b. Each of the grooves 51b and 53b are equidistant from each other.

The grooves 51b and 53b may have various shapes such as but not limited to rectangular, circular, square etc. In an embodiment, the grooves 51b and 53b are rectangular shaped.

The ring 51a may have a diameter ranging from 4 mm to 8 mm. In an embodiment, the diameter of the ring 51a is 6 mm. The nut 53a may have a diameter ranging from 2 mm to 8 mm. The pitch of the nut 53a may be in a range of 0.3 mm to 0.8 mm. In an embodiment, the pitch of the nut 53a is 0.5 mm.

The crown 60 may be manufactured in a predefined shape but not limited to L-shaped, W-shaped, etc. In an embodiment, the crown 60 is manufactured in L shape as depicted in FIG. 1b. The L-shaped design of the crown 60 reduces stress on the implant 100, thus preventing damage on the native annulus of the mitral valve and helps in smooth actuation of the mitral valve. Further, the L-shape of the crown 60 may help in assembling the device in a simplified manner.

The implant 100 may include a predefined number of crowns 60. The predefined number of crowns 60 may be in a range of six to ten. In an embodiment, the implant 100 includes seven crowns 60. The crown 60 may include at least two arms i.e., a primary arm 61 and a secondary arm 63. The crown 60 may be used to assemble anchors 70, fastener 50 and the nut 53a. Further, the crown 60 may be used to perform expansion and compression of the implant 100 at the implantation site. The primary arm 61 may be longer than the secondary arm 63 in a predefined ratio. In an embodiment, the primary arm 61 is twice the length of the secondary arm 63. The primary arm 61 may include a proximal end 61a, a distal end 61b and a length L2 extending between the primary arm 61 and the secondary arm 63. The length L2 may be in a range of 25 mm to 32 mm. In an embodiment, the length L2 is 28 mm.

The secondary arm 63 may bifurcate at a predefined length L3 from the distal end 61b of the primary arm 61. The length L3 may be in a range of 4 mm to 8 mm. In an embodiment, the length L3 is 6 mm. The secondary arm 63 may include a proximal end 63a, a distal end 63b and a length L4 extending between the proximal end 63a and the distal end 63b. The length L4 may be in a range of 10 mm to 15 mm. In an embodiment, the length L4 is 12 mm.

In yet another embodiment, the secondary arm 63 is longer than the primary arm 61.

The primary arm 61 and the secondary arm 63 may be straight or curved. In an embodiment, the primary arm 61 and the secondary arm 63 are slightly curved having a radius of curvature ranging from 18 mm to 25 mm. In an embodiment, the radius of curvature is 21 mm. The two arms of the crown 60 may have a predefined thickness ranging from 300 microns to 750 microns. In an embodiment, the thickness is 550 microns.

The crown 60 may be assembled with the fastener 50 by means of the grooves 51b and 53bprovided in the ring 51a and the nut 53a respectively. The crown 60 may be secured by means of without limitation, welding, adhesion, snap fix mechanism etc. In an embodiment, the crown 60 is secured by means of the welding. The primary arm 61 may be attached to the ring 51a and the secondary arm 63 may be attached to the nut 53a as explained below.

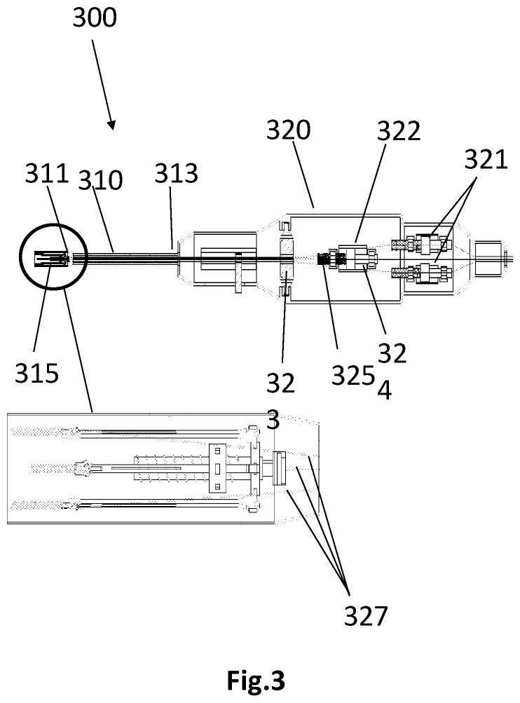

The primary arm 61 may include a first connecting structure 62 at the proximal end 61a. The first connecting structure 62 may be shaped such as but not limited to a beak shaped structure, T-shaped structure, etc. In an embodiment, the first connecting structure 62 is shaped in the beak shaped structure. The first connecting structure 62 maybe fitted inside the plurality of first grooves 51b of the ring 51a of fastener 50. Further, the secondary arm 63 may be fitted inside the plurality of second grooves 53b of the nut 53a. Further, the primary arm 61 may include a second connecting member 64 at the distal end 61b as depicted in FIG. 1. The second connecting member 64 may be used to assemble the anchors 70 with crown 60. The second connecting structure 64 may be manufactured in a predefined shape such as but not limited to, a rectangular shape, square shape, oval shape, etc. In an embodiment, the second connecting member 64 is rectangular shaped. The second connecting member 64 may include a plurality of slots (not shown) to attach the anchor 70 on the crown 60 as illustrated in FIG. 3. The plurality of slots may be used to provide smooth movement to the anchor 70 at the time of actuation of the implant 100.

The crown 60 may be made of a biocompatible metallic material but not limited to SS (stainless steel) alloy, nitinol alloy (TiNi-SS, TN3, TNC, TiNi-YY, Ti-Ni-01, and Ti-Ni-02) and CoCr (cobalt chromium) alloy. In an embodiment, the crowns 60 of the implant 100 are made of nitinol alloy due to its super elasticity and shape memory property.

The anchor 70 may be a resilient member such as spring, a coil etc. In an embodiment, the anchor 70 is a coil. The implant 100 may include a predefined number of the anchors 70 ranging from six to ten. In an embodiment, the implant 100 includes 9 anchors. The anchor 70 may facilitate anchoring of the implant 100 on the annulus of the mitral valve. The anchor 70 may be manufactured by coiling a wire of diameter ranging between 0.1 mm to 0.4 mm. In an embodiment, the diameter is 0.2 mm. The anchor 70 may have a predefined pitch ranging from 0.5 mm to 3 mm. In an embodiment, the pitch of the anchor 70 is 1 mm.

The anchor 70 may have a proximal end 71, a distal end 73 and a length L5 extending between the proximal end 71 and the distal end 73. The length L5 may be in a range of 5 mm to 20 mm. In an embodiment, the length L5 is 12 mm. The length L5 may vary as per the thickness of the native annulus of the mitral valve.

The anchor 70 may be attached with the crown 60. In an embodiment, the distal end 61b of the primary arm 61 is attached with the proximal end 71 of the anchor 70. The anchor 70 may be manually attached with the crown 60. Further, the distal end 73 of the anchor 70 may have a sharpened edge which may facilitate effective penetration of the implant 100 on the tissue wall of the native mitral valve with minimum damage to the tissue in order to facilitate anchoring of the implant 100. Alternatively, the distal end 73 includes a hook (not shown) which may facilitate penetration of the anchor 70 in the tissue wall.

The anchor 70 may be made of a metallic material but not limited to 316L SS, 316LVM SS, 304 SS. In an embodiment, the anchor 70 is made of 316L SS.

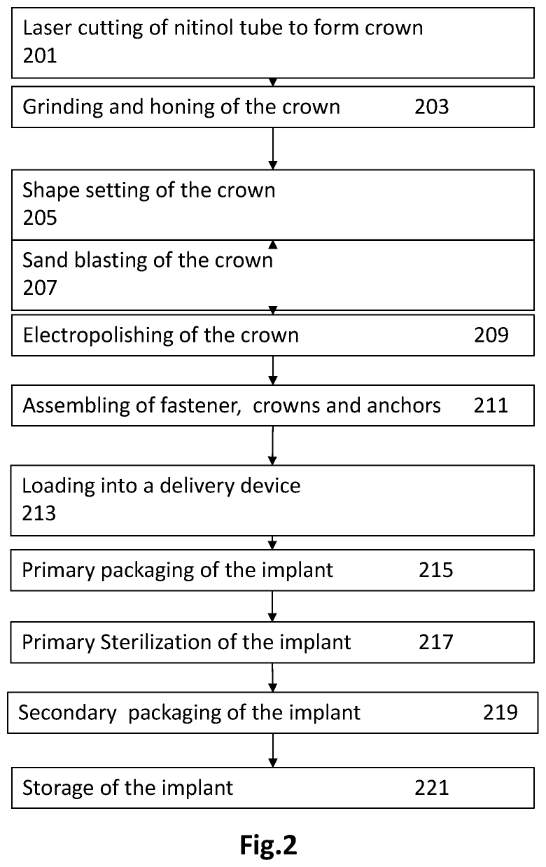

In accordance with the present invention, FIG. 2 depicts a flow chart of a process involved in manufacturing of the implant 100. The process commences at a step 201. At step 201, a tube made of nitinol is subjected to a process of cutting to form the crown 60. The nitinol tube may be an oxide free tube. The cutting may be performed by any means such as without limitation liquid jet machining, laser cutting, abrasive jet machining, etc. In an embodiment, the cutting of the nitinol tube is performed by means of laser cutting. The tube may have an outer diameter in a range of 4 mm to 6 mm. In an embodiment, the outer diameter of the tube is 5.5 mm. Further, the tube may have a predefined wall thickness ranging from 0.40 mm to 0.90 mm. In an embodiment, the thickness of the tube is 0.6 mm.

The laser cutting may be performed with gas such as but not limited to argon gas, or oxygen gas. In an embodiment, the laser cutting is performed with argon gas. The pressure of argon gas may be maintained in a range of 2 bars to 18 bars.

At step 203, the crown 60 obtained at previous step are subjected to a process of grinding and honing to produce a smooth surface. The process of grinding may be performed by means of diamond and/or abrasive files. In an embodiment, honing is performed by means of using abrasive stones of 3.4 mm to 3.5 mm. Alternatively, an abrasive gel may also be used to remove burrs generated during the laser cutting process.

At step 205, the crown 60 is subjected to a process of shape setting post grinding and honing at the previous step. The proce ss of shape setting may be performed by means of heat treatment using a mold. The process of shape setting is performed in multiple steps. Firstly, the laser cut crown 60 are expanded from a crimped diameter ranging from 3 mm to 5 mm to an expanded diameter ranging from 20 mm to 45 mm using a mandrel. Post expansion, the crown 60 may be subjected to a process of moulding to impart a predefined radius of curvature to the crown 60. The process of moulding is performed in three steps. The process of moulding may be performed at a temperature of 505° C. for 10 minutes to 15 minutes. In an embodiment, the moulding is performed at a temperature of 505° C. for time duration of 15 minutes.

At step 207, the crown 60 is subjected to a process of sand blasting. The process of sand blasting may be performed by means of aluminium oxide powder. The aluminium oxide powder is allowed to strike on the crown 60 at a predefined frequency for a predefined period of time. The predefined frequency and the predefined time may be in a range of 45 to 85 Hz and 3 minutes to 10 minutes respectively. In an embodiment, the frequency is in range of 55 to 65 Hz and time is 5 minutes to 8minutes. The pressure exerted by the powder may vary in a range of 20 psi to 90 psi, preferably in a range of 30 psi to 60 psi. The process of sand blasting may produce highly smooth outer surface of the crown 60 due to abrasion with the aluminium powder. The process of sand blasting may also remove oxide layers, striations left by laser cutting in previous step, decrease propensity for micro cracking and/or provide light texture to the outer surface of the crown 60.

Lastly, at step 209, the crown 60 is subjected to a process of electro-polishing. The process of electro-polishing is performed by immersing the implant 100 in a solution of electrolytes and passing the current at a predefined voltage for a predefined time duration. The solution of electrolytes may be a concentrated acid solution such as a mixture of 20% to 25% purfluoric acid and 75% to 80% acetic acid. The voltage used for electro polishing is from 8V to 12V, and the current is from 0.3 A to 1.5 A, preferably from 0.8 A to 0.10 A. The predefined time duration is in a range of 1 minute to 8 minutes, preferably 2 to 6 mins.

At step 211, the components such as the fastener 50, a plurality of crowns 60, a plurality of anchors 70 are assembled together to form the implant 100 as depicted in FIG. 1.

At step 213, the implant 100 is loaded onto a delivery device 300 as depicted in FIG. 3.

The delivery device 300 may include a shaft 310 and a body 320. The shaft 310 may include a proximal end 311 and a distal end 313. The shaft 310 may include a head 315 at the proximal end 311.

The body 320 may include one or more rollers, for example, a set of proximal rollers 321, a distal roller 322, and at least one guide plate 323. The proximal rollers 321 and the distal rollers 322 of the delivery device 300 are attached to the anchor 70 and the fastener 50 of the implant 100 respectively. The proximal roller 321 may include two shafts, for example, a plain shaft 324 and a threaded shaft 325. The threaded shaft 325 may provide rotational movements in clockwise & counter clockwise direction. The plain shaft 324 of the proximal roller 321 provides axial movement in forward and backward direction.

The proximal rollers 321 are connected to the anchors 70 with the help of wires 327 at the hook of the anchors 70 in order to fix the implant 100 on the tissue annulus. The distal rollers 322 are connected to the top portion 52 with the help of the wires 327 in order to rotate the fastener 50 for actuating the implant 100. The guided plate 323 is placed between the proximal rollers 321, distal rollers 322 and the implant 100, which is mainly used to guide the wires 327 to the components and provide individual movement to the implant 100.

At step 215, the implant 100 is subjected to a process of primary packaging. The implant 100 with the delivery device 300 may be packaged in a pouch made of aluminium. Similarly, the implant 100 with the delivery device 300 may be packaged in the pouch made of aluminium.

Post primary packaging, the implant 100 is subjected to a process of primary sterilization at step 217. The process of primary sterilization may be performed by means of gas sterilization using gases such as ETO (Ethylene oxide) gas, nitrogen gas. In an embodiment, the process of primary sterilization is performed by means of ETO (Ethylene oxide) gas.

At step 219, the implant 100 is subjected to a process of secondary packaging. During the process of secondary packaging, the implant 100 may be packaged in a plastic box, cardboard box, etc. In an embodiment, the implant 100 is packaged in a plastic box.

Post-secondary packaging, the implants 100 may be stored at a predefined temperature at step 221. In an embodiment, the implant 100 is stored at the room temperature.

The present invention will now be explained with the help of following examples.

The present invention will now be explained with the help of following examples.

Example 1 (Present invention): An in-vitro tissue model of the mitral valve including leaflets of size 36 mm initial diameter was used. The implant having 40 mm diameter in a conical expandable configuration was prepared. The implant was placed over the leaflets of the mitral valve and anchored with the help of anchors. Once the anchors were positioned and fixed onto the leaflets of the in-vitro tissue model, the implant was actuated by rotating a singular fastener. As the fastener was rotated with the help of a spanner, a nut moved upwards resulting in crimping of the implant and the leaflet diameter decreased from 36 mm to 30 mm. The time taken for the whole procedure was 20 minutes in In-vitro model with effective manner.

Example 2: An in-vitro tissue model of the mitral valve including leaflets of size 34 mm initial diameter was used. An implant of diameter 38 mm, having V shaped multiple crowns and multiple fasteners was used at the treatment site. It was found that the implant V shaped crowns were subjected to more stress at the treatment site. Also, the time taken to repair the valve was comparatively more than the present invention.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. An implant (100) for treatment of regurgitation in a mitral valve, the implant (100) comprising:
at least one fastener (50) including a top portion (52) and a shank (54), the top portion (52) including a ring (51a); and
a plurality of crowns (60) including a primary arm (61) and a secondary arm (63), the primary arm (61) being longer than the secondary arm (63);
wherein the ring (51a) of the top portion (52) is attached at a proximal end (51) of the shank (54) and a nut (53a) attached at a distal end (53) of the shank (54),
wherein the primary arm (61) is attached to the ring (51a) and the secondary arm (63) is attached to the nut (53a), and
wherein the top portion (52) is rotated in a predefined direction leading to upward movement of the nut (53a) resulting in radial contraction of the crowns (60) in order to actuate an implant (100) at an implantation site.

2. The implant (100) for treatment of regurgitation in a mitral valve as claimed in claim 1 wherein the ring (51a) is fixedly attached to shank (54).

3. The implant (100) for treatment of regurgitation in a mitral valve as claimed in claim 1 wherein the nut 53a is removably attached with the shank (54).

4. The implant (100) for treatment of regurgitation in a mitral valve as claimed in claim 1 wherein the plurality of crowns (60) includes six to ten crowns.

5. The implant (100) for treatment of regurgitation in a mitral valve as claimed in claim 1 wherein the primary arm (61) is twice the length of the secondary arm (63).

6. The implant 100 for treatment of regurgitation in a mitral valve as claimed in claim 1 wherein the primary arm (61) and the secondary arm (63) are slightly curved.

7. The implant (100) for treatment of regurgitation in a mitral valve as claimed in claim 1 wherein the ring (51a) and the nut (53a) includes a plurality of grooves (51b) and (53b).

8. The implant 100 for treatment of regurgitation in a mitral valve as claimed in claim 1 wherein the primary arm (61) includes first connecting structure (62) at a proximal end (61a).

9. The implant 100 for treatment of regurgitation in a mitral valve as claimed in claim 1 wherein a plurality of anchors (70) is provided to fix the implant (100) at the implantation site.

10. The implant (100) for treatment of regurgitation in a mitral valve as claimed in claim 9 wherein the plurality of anchors (70) includes six to ten anchors.
